(19) United States
(12) Patent Application Publication
(10) Pub. No.: US 2010/0036221 A1
(43) Pub. Date: Feb. 11, 2010

(54) Noninvasive Method to Estimate Variation of Blood Glucose Levels Using Metabolic Measurements

(76) Inventors: Chiyeung Lee, Hong Kong (CN); Yingsheung Cheung, Hong Kong (CN); Karen Siuling Lam, Hong Kong (CN); Victor Onkwok Li, Hong Kong (CN)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/519,322

(22) PCT Filed: Jan. 10, 2008
(86) PCT No.: PCT/CN08/00064

§ 371 Date: Oct. 9, 2009

Related U.S. Application Data
(60) Provisional application No. 60/881,265, filed on Jan. 19, 2007.

(51) Int. Cl. A61B 5/145

(52) U.S. Cl. 600/365

(57) ABSTRACT

A method to estimate the variation of blood glucose concentration in a patient without blood sampling. The method can be implemented by measuring metabolic parameters including heat dissipation by conduction, percentage oxygen content of expired air and volume per minute of expired air. These parameters are used to calculate estimated blood glucose variation.

### FIELD OF THE INVENTION

This invention relates to a method for measuring the variation of human blood glucose concentration without blood sampling, and more particularly to a method that uses measurable body parameters to estimate the variation in blood glucose concentration.

### BACKGROUND OF THE INVENTION

The amount of glucose in blood directly affects the glucose oxidation rate. It has been shown that hyperglycemia significantly increases the carbohydrate (CHO) oxidation rate in normal and type 2 diabetic human beings [1, 2, 3].

Glucose oxidation is an exothermic chemical reaction. Biological glucose oxidation produces heat energy [4]. This probably explains the experimental results indicating facial and sublingual temperature rises after intravenous glucose injection in diabetic subjects [5].

It has been discovered that human energy expenditure increases after oral glucose load [6]. A Weir equation was derived to determine metabolic rate, which is a measure of human energy expenditure [7]. Later, an alternative version of the Weir equation was provided using data that focus on glucose oxidation [8]. The equation indicates that metabolic rate depends on percentage oxygen content of expired air and volume per minute of expired air.

A metabolic heat conformation (MHC) method for noninvasive blood glucose measurement has been proposed [9, 10], [11, 12, 13]. This method makes use of thermal and optical techniques to measure body glucose metabolic effects at an extremity's tip, such as a forefinger tip. A multiple linear regression equation was formed based on the measured parameters. The apparatus was designed by hard-coding the regression equation to a ROM unit based on the measured results of only 8 subjects (2 normal+6 diabetic). The population of sample subjects is small, so that variations of the measured parameters due to variations of body properties (e.g., skin thickness), among different subjects were not indicated. Thus, the results are far from conclusive, and do not appear to constitute an accurate predictive model from the measured parameters.

Therefore, a novel noninvasive reliable and accurate method to estimate the variation of human blood glucose concentration is needed.

### SUMMARY OF THE INVENTION

The present invention provides methods of estimating the variation of blood glucose in a subject without blood sampling. In a preferred embodiment, the subject invention provides a method that can be implemented by measuring metabolic parameters related to glucose oxidation including, for example, heat dissipation, oxygen content of expired air, and the rate of air expiration (e.g. volume per minute of expired air).

A further aspect of the subject invention is the development of a classification model based on measurements from a clinical trial. The methods of the subject invention obtain results that are sufficiently accurate to grade the variation of blood glucose into, for example, five classes according to the classification model, without blood sampling. Based on this model, estimation of blood glucose variation can be performed. Advantageously, the variation of blood glucose can be accurately determined using the method of the present invention.

A further aspect of the subject invention is a device that facilitates taking simple metabolic measurements in order to implement the method of the subject invention.

Owing to its noninvasive and easy-to-use features, self-monitoring of blood glucose (SMBG) according to the subject invention is made possible for home use. Thus, not only can this method lead to better control of blood glucose level, but it can also help facilitate a healthy lifestyle for normal, pre-diabetic and type 2 diabetic persons.

### BRIEF DESCRIPTION OF THE DRAWINGS

Further features and advantages of the invention will become apparent upon review of the following detailed description of the preferred embodiments thereof in conjunction with the drawings in which:

FIG. 1 is a schematic diagram of the method of the present invention;

FIG. 2 shows the calibration process of the method of FIG. 1;

FIG. 3 shows the estimation process of blood glucose variation;

FIG. 4 presents a scattered plot of the estimated blood glucose variation against the reference blood glucose variation using multiple linear regression analysis; and

FIG. 5 presents the classification results by the inventive method using 3 regions.

### DETAILED DESCRIPTION OF THE PREFERRED EMBODIMENTS

In accordance with the subject invention, it is possible to analyze and determine the extent of blood glucose variation in a patient using noninvasive metabolic measurements. The measurements used according to the subject invention, optionally used in conjunction with the classification model described herein, is able to obtain results with sufficient accuracy for home-used blood glucose monitoring. The methods and devices of the subject invention can be used to monitor blood glucose variation and/or for lifestyle education for normal, pre-diabetic and type 2 diabetic persons.

The present invention provides methods of estimating the variation of blood glucose in a subject without blood sampling. In a preferred embodiment, the subject invention provides a method that can be implemented by measuring metabolic parameters related to glucose oxidation including, for example, heat dissipation, the oxygen content of expired air, and the rate of air expiration.

Heat dissipation may be measured at an extremity. Preferably heat dissipation is measured at a fingertip. In a preferred embodiment, the oxygen content is of air expired by the patient is measured as a percentage of the gases expired by the patient. Furthermore, preferably, the rate of air expiration is measured as a volume of air expired per minute.

A further aspect of the subject invention is the development of a classification model based on measurements from a clinical trial. The methods of the subject invention obtain results that are sufficiently accurate to grade the variation of blood glucose into, for example, five classes according to the classification model, without blood sampling. Based on this model, estimation of blood glucose variation can be performed. Advantageously, the variation of blood glucose can be accurately determined using the method of the present invention.

A further aspect of the subject invention is a device that facilitates taking simple metabolic measurements in order to implement the method of the subject invention.

Owing to its noninvasive and easy-to-use features, self-monitoring blood glucose (SMBG) according to the subject invention is made possible for home use. Thus, not only can this method lead to better control of blood glucose level, but it can also help facilitate a healthy lifestyle for normal, pre-diabetic and type 2 diabetic persons.

The subject invention further pertains to devices whereby the methods of the subject invention can be carried out utilizing a portable or non-portable apparatus that measures parameters and/or makes calculations relevant to blood glucose concentration. The device of the subject invention can be used by a patient, remain at a patient's home, or be in a physician's office, a laboratory or hospital. The device can measure, for example, heat dissipation, the oxygen content of expired air and/or the rate of air expiration.

Blood glucose is a fuel to produce necessary energy for living bodies. Such energy is produced through a chemical reaction called glucose oxidation, which can be simply expressed in the following chemical equation (1):

C₆H₁₂O₆+6O₂→6CO₂+6H₂O+36ATP (1)

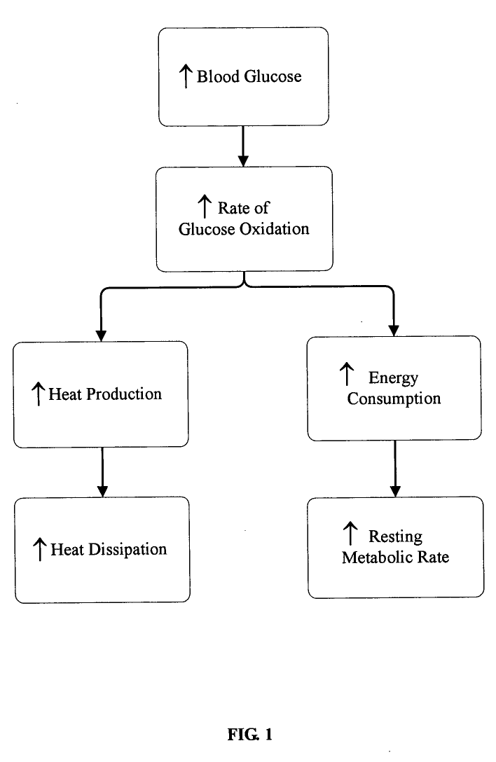

In glucose oxidation, oxygen in blood is consumed and heat is produced (ΔH=−686 kcal/mol). Therefore, oxygen is consumed for energy production. When the concentration of blood glucose increases, the rate of glucose oxidation also increases. This, in turn, causes an increase both heat dissipation and energy expenditure, which can be measured by resting metabolic rate (RMR). These biological relationships are shown in FIG. 1.

RMR can be calculated using the revised Weir equation (2):

RMR=(1.039−0.05Oₑ)·V (2)

where Oₑ is percentage oxygen content of expired air, and V is volume per minute of expired air.

Based on the concept of glucose metabolism, a method has been proposed to estimate blood glucose variation noninvasively with the following assumptions:
1. Blood glucose can be estimated based on heat production and energy expenditure;
2. The amount of heat production and the amount of heat dissipation are equal;
3. The amount of heat dissipation can be determined at an extremity's tip by a conduction method;
4. Energy expenditure is represented by RMR, which is dependent on Oₑ and V;
5. Measurements are taken from the subjects under resting condition;
6. Artifacts are avoided before measurements; and
7. Subjects do not have fever, hand trauma and respiratory diseases.

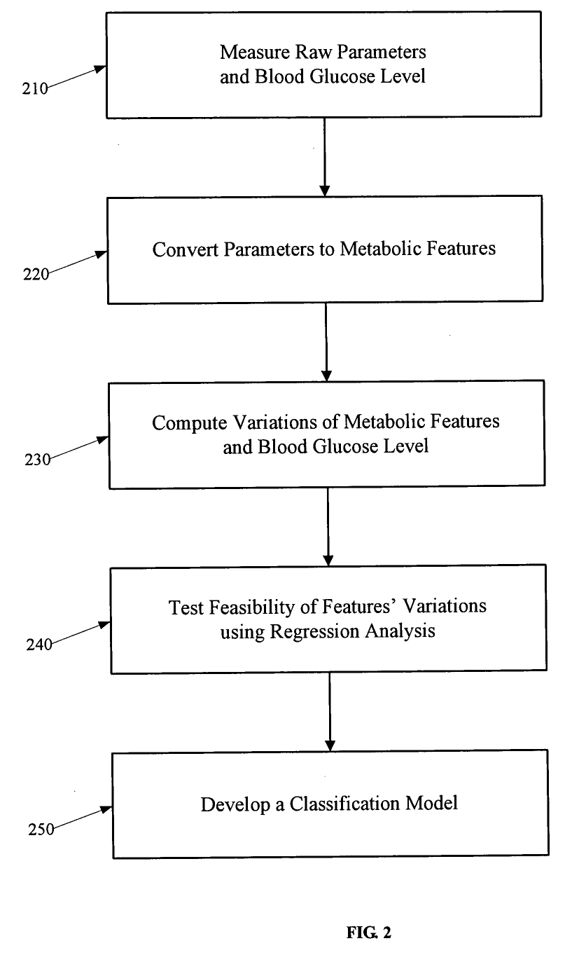

FIG. 2 shows the steps of the calibration process. In step 210, raw metabolic parameters (i.e. conduction heat loss at the extremity's tip, Oₑ and V) are measured. Blood glucose level is also measured using finger-pricking method for reference purpose. In step 220, the measured parameters are converted to metabolic features (i.e. heat dissipation by conduction and RMR). In step 230, for each subject, variations of metabolic features and variation of blood glucose level are computed by subtracting the previously measured values. This can account for the variations of different subjects since they have different biological properties and thus, different offset metabolic values. In step 240, the variations of metabolic features are tested whether they are feasible to become features of the classification model. Multiple linear regression analysis using least-squares method is carried out. In step 250, the feasible variations of features and the corresponding reference blood glucose variation are used to train and develop a classification model for future estimation of blood glucose variation. Linear discriminant classifier is adopted to obtain the best performance and classification accuracy.

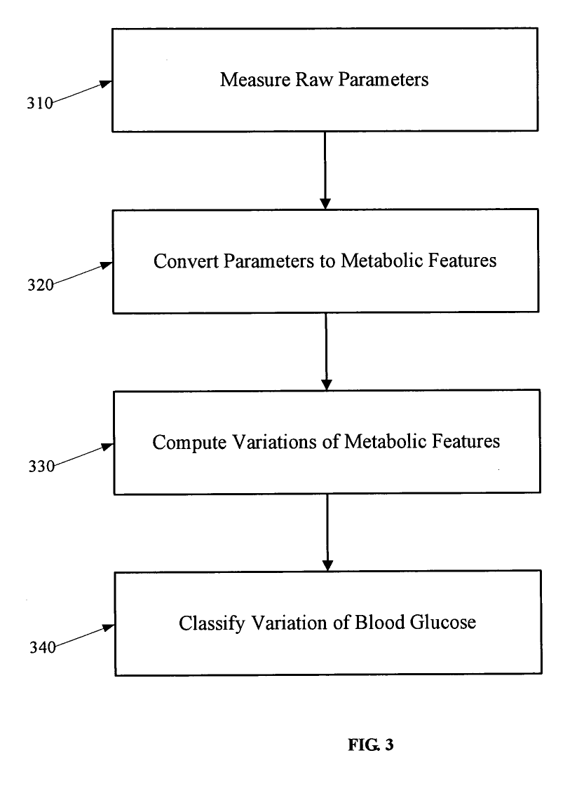

FIG. 3 describes the estimating process of blood glucose variation once the classification model has been developed. In step 310, conduction heat flow, Oₑ and V are measured. In step 320, these measured parameters are converted to heat dissipation by conduction and RMR. In step 330, for each subject, variations of metabolic features are computed by subtracting the previously measured values in order to account for subject variability. In step 340, the variations of metabolic features are input to the classifier. After classification, the extent of blood glucose variation is obtained.

Multiple linear regression using least-squares method is adopted to test the feasibility of features' variation to become features of the classification model. Suppose that n is number of samples taken, X₁ is variation of heat dissipation by conduction, X₂ is RMR variation, y is reference blood glucose variation, Y is estimated blood glucose variation and e is the error with respect to a measured value yₖ (k=1, . . . , n) using finger-pricking method, the regression equation (3) is written such that values of coefficient aᵢ (i=0,1,2) are going to be determined using least-squares method:

y=a₀+a₁X₁+a₂X₂+e (3)

Since n samples are taken, equation (3) is expanded to n equations, which can be summarized with equation (4):

y=Xa+e (4)

where y is a n×1 vector consisting of yₖ's (k=1, . . . , n), X is a n×3 matrix consisting of [1 X₁ₖ X₂ₖ] (k=1, . . . , n), a is a 3×1 vector which is equal to [a₀ a₁ a₂]^{t} and e is a n×1 vector consisting of eₖ's (k=1, . . . , n).

Let Jₛ(a) be the sum-of-squared-error criterion function with respect to a:

$${J}_{s} ( a ) = {‖ e ‖}^{2} = {‖ y - Xa ‖}^{2} = {\sum }_{i = 1}^{n} {( {y}_{i} - {a}^{t} {X}_{i} )}^{2} \qquad ( 5 )$$

The first-order derivative of equation (5) is expressed in equation (6):

$$∇ {J}_{s} = - {\sum }_{i = 1}^{n} 2 ( {y}_{i} - {a}^{t} {X}_{i} ) {X}_{i} = 2 {X}^{t} ( Xa - y ) \qquad ( 6 )$$

To minimize the sum-of-squared-error criterion function, its first-order derivative is set to be zero. Therefore, a can be solved using equation (7):

∇Jₛ=0

[symbol]

2X^{t}(Xa−y)=0

[symbol]

X^{t}Xa=X^{t}y

[symbol]

a=(X^{t}X)⁻¹X^{t}y (7)

As a result, Y, a n×1 vector consisting of estimated values Yₖ's (k=1, . . . , n), can be calculated using equation (8).

Y=Xa (8)

A classification model is designed and carried out using linear discriminant analysis (LDA). Assume the sample points are normally distributed, Bayes formula is defined in equation (9) where x is a d-component column vector, ωᵢ represents the state of nature for region i (Rᵢ) such that Rᵢ and Rⱼ are adjacent to each other, c is the number of regions, P(ωᵢ|x) is the posterior probability, p(x|ωᵢ) is the likelihood, P(ωᵢ) is the prior probability and p(x) is the evidence factor defined in equation (10). Equation (11) expresses the general multivariate normal likelihood in d dimensions where μᵢ is the d-component mean vector with respect to region i and Σᵢ is the d-by-d covariance matrix with respect to region i:

$$\begin{matrix}P ( {ω}_{i} \vert x ) = \frac{p ( x \vert {ω}_{i} ) P ( {ω}_{i} )}{p ( x )} for i = 1 , 2 , … , c \\ where & ( 9 ) \\ p ( x ) = {\sum }_{k = 1}^{c} p ( x \vert {ω}_{k} ) P ( {ω}_{k} ) \\ and & ( 10 ) \\ p ( x \vert {ω}_{i} ) = \frac{1}{{( 2 π )}^{\frac{d}{2}} {\vert {Σ}_{i} \vert}^{\frac{1}{2}}} exp [ - \frac{1}{2} {( x - {μ}_{i} )}^{t} {Σ}_{i}^{- 1} ( x - {μ}_{i} ) ] \\ for i = 1 , 2 , … , c & ( 11 )\end{matrix}$$

According to the Bayes decision theory, the classification decision depends on the discriminant function g(·) defined below:

$$\begin{matrix}\begin{matrix} Decide {ω}_{i} if P ( {ω}_{i} \vert x ) > P ( {ω}_{j} \vert x ) & ∀ j \neq i\end{matrix} & ( 12 ) \\ \begin{matrix} ⇒ Decide {ω}_{i} if \frac{p ( x \vert {ω}_{i} ) P ( {ω}_{i} )}{p ( x )} > \frac{p ( x \vert {ω}_{j} ) P ( {ω}_{j} )}{p ( x )} & ∀ j \neq i\end{matrix} & \\ \begin{matrix} ⇒ Decide {ω}_{i} if p ( x \vert {ω}_{i} ) P ( {ω}_{i} ) > p ( x \vert {ω}_{j} ) P ( {ω}_{j} ) & ∀ j \neq i\end{matrix} & \\ \begin{matrix}⇒ Decide {ω}_{i} if ln p ( x \vert {ω}_{i} ) + ln P ( {ω}_{i} ) > ln p ( x \vert {ω}_{j} ) + ln P ( {ω}_{j} ) & ∀ j \neq i\end{matrix} & \\ \begin{matrix} ⇒ Decide {ω}_{i} if {g}_{i} ( x ) > {g}_{j} ( x ) & ∀ j \neq i\end{matrix} \\ where \{ \begin{matrix}{g}_{i} ( x ) = ln p ( x \vert {ω}_{i} ) + ln P ( {ω}_{i} ) \\ {g}_{j} ( x ) = ln p ( x \vert {ω}_{j} ) + ln P ( {ω}_{j} )\end{matrix} & \end{matrix}$$

Substitute equation (11) into gᵢ(x) in equation (12) becomes:

$$\begin{matrix}{g}_{i} ( x ) = ln p ( x \vert {ω}_{i} ) + ln P ( {ω}_{i} ) \\ = - \frac{1}{2} {( x - {μ}_{i} )}^{t} {Σ}_{i}^{- 1} ( - {μ}_{i} ) - \frac{d}{2} ln ( 2 π ) - \frac{1}{2} ln \vert {Σ}_{i} \vert + ln P ( {ω}_{i} )\end{matrix} \qquad ( 13 )$$

Assume all the regions have identical covariance matrices (i.e. Σᵢ=Σ), the terms that are independent of i are eliminated. Equation (13) thus becomes:

$$\begin{matrix}{g}_{i} ( x ) = - \frac{1}{2} {( x - {μ}_{i} )}^{t} {Σ}^{- 1} ( x - {μ}_{i} ) \underbrace{- \frac{d}{2} ln ( 2 π ) - \frac{1}{2} ln \vert Σ \vert}_{Independent of i} + ln P ( {ω}_{i} ) \\ = - \frac{1}{2} {( x - {μ}_{i} )}^{t} {Σ}^{- 1} ( x - {μ}_{i} ) + ln P ( {ω}_{i} ) \\ = - \frac{1}{2} ( \underbrace{{x}^{t} {Σ}^{- 1} x}_{Independent of i} - 2 {μ}_{i}^{t} {Σ}^{- 1} x + {μ}_{i}^{t} {Σ}^{- 1} {μ}_{i} ) + ln P ( {ω}_{i} ) \\ = {μ}_{i}^{t} {Σ}^{- 1} x - \frac{1}{2} {μ}_{i}^{t} {Σ}^{- 1} {μ}_{i} + ln P ( {ω}_{i} ) \\ = {w}_{i}^{t} x + {ω}_{i 0}\end{matrix} \\ where \\ {w}_{i} = {Σ}^{- 1} {μ}_{i} and {ω}_{i 0} = - \frac{1}{2} {μ}_{i}^{t} {Σ}^{- 1} {μ}_{i} + ln P ( {ω}_{i} ) \qquad ( 14 )$$

The calculation of gⱼ(x) is similar. A linear decision boundary can therefore be obtained as follows:

$${g}_{i} ( x ) = {g}_{j} ( x ) \\ ⇒ {w}_{i}^{t} x + {ω}_{i 0} = {w}_{j}^{t} x + {ω}_{j 0} \\ ⇒ ( {w}_{i}^{t} - {w}_{j}^{t} ) x + ( {ω}_{i 0} - {ω}_{j 0} ) = 0 \\ ⇒ {w}^{t} x + {ω}_{0} = 0 \\ where \{ \begin{matrix}w = {w}_{i} - {w}_{j} = {Σ}^{- 1} ( {μ}_{i} - {μ}_{j} ) \\ {ω}_{0} = {ω}_{i 0} - {ω}_{j 0}\end{matrix} \qquad ( 15 )$$

### Trial Design

The apparatus used are all noninvasive and commercially available. They are as follows:
1. Data Harvest EasySense Advanced Datalogger, which was used to measure conduction heat loss at the extremity's tip;
2. Teledyne AX300 Oxygen Analyzer with R-17 MED Oxygen Sensor, which was used to measure percentage oxygen content of expired air at the mouth cavity;
3. Vitalograph Micro Spirometer, which was used to measure volume per minute of expired air; and
4. Medisense Optium Xceed Meter, which was used to measure blood glucose level for calibration and reference purposes.

The clinical trial was done with informed consent by the subjects and ethical approval by the Institutional Review Board of the University of Hong Kong/Hospital Authority Hong Kong West Cluster (HKU/HA HKW IRB) at Queen Mary Hospital (QMH). A total of 190 subjects (31 normal and 159 type 2 diabetic), aged from 23 to 86, participated in this trial.

The trial protocol is as follows:
1. Patient should report to the clinic fasting (at least 12 hours, no food or drink except water), having not taken their morning study medication dose.
2. Patient should sit down and rest for 15 minutes.
3. Patient should have a blood glucose measurement taken as usual.
4. Patient should have a conduction heat loss measurement taken.
5. Patient should have a percentage oxygen content of expired air measurement taken.
6. Patient should have a volume per minute of expired air measurement taken.
7. The patient should complete eating a meal (standard meal is not necessary).
8. Step 3 to 6 should be repeated 45 minutes after the start of the meal.

### Results

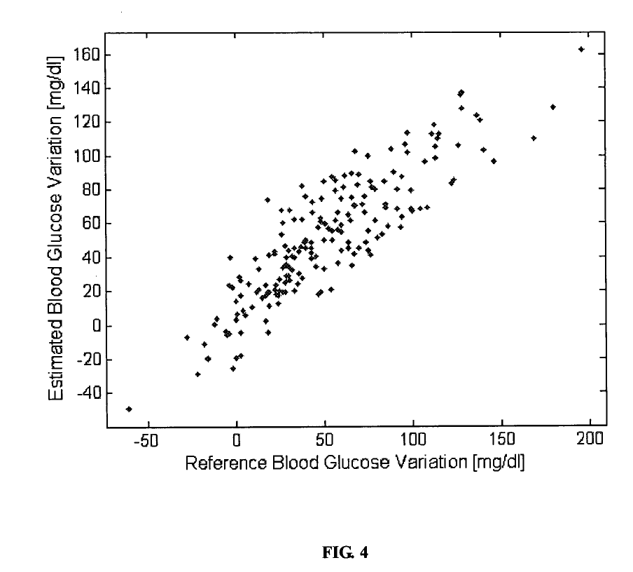

FIG. 4 presents the scattered plot of the estimated blood glucose variation (Y) versus the reference blood glucose variation (y) using multiple linear regression analysis where n=190. It can be seen that a good correlation is obtained with correlation coefficient (R) equals to 0.88. Thus, variation of heat dissipation by conduction and RMR variation can be used as features of the classification model.

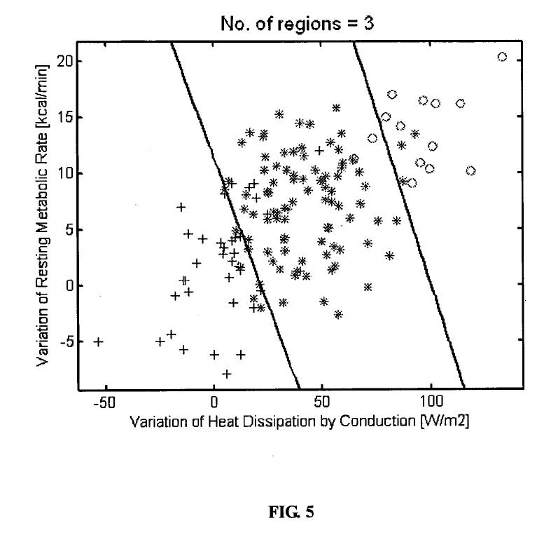

The classifier has been tested 100 times by randomly choosing half of the samples as training set and half of them as testing set. FIG. 5 presents the LDA classification results using 3 regions. The classification accuracy is 84.26%. When 4 and 5 regions were used, the classification accuracy is 71.98% and 71.82% respectively. The result shows that the method of the subject invention can be used to estimate blood glucose variation.

Having thus described at least illustrative embodiments of the invention, various modifications and improvements will readily occur to those skilled in the art and are intended to be within the scope of the invention. Accordingly, the foregoing detailed description is by way of example only and is not intended as limiting. The invention is limited only as defined in the following claims and the equivalents thereto.

### REFERENCES

1. Chevaux et al., “Study by Indirect Calorimetry of the Oxidation Rate of Carbohydrate in Man at Two Different Plasma Insulin Levels” (abstract), Diabetologia, 12, 383 (1976).
2. Felber et al., “Carbohydrate and Lipid Oxidation in Normal and Diabetic Subjects”, Diabetes, 26, 693-699 (1977).
3. Meyer et al., “Modifications of Glucose Storage and Oxidation in Nonobese Diabetics, Measured by Continuous Indirect Calorimetry”, Diabetes, 29, 752-756 (1980).
4. Scott, “Contribution of anaerobic energy expenditure to whole body thermogenesis”, Nutrition & Metabolism, 2, 14 (2005).
5. Hillson et al., “Facial and sublingual temperature changes following intravenous glucose injection in diabetics”, Diabetes & Metabolism, 8, 15-19 (1982).
6. Rousselle et al., “Relationship Between Glucose Oxidation and Glucose Tolerance in Man”, Metabolism, 31(9), 866-870 (1982).
7. Weir, “New methods for calculating metabolic rate with special reference to protein metabolism”, The Journal of Physiology, 109, 1-9 (1949).
8. Mansell et al., “Reappraisal of the Weir equation for calculation of metabolic rate”, American Journal of Physiology, 258(6 Pt 2), R1347-1354 (1990).
9. Ko et al., “Body Metabolism Provides a Foundation for Noninvasive Blood Glucose Monitoring”, Diabetes Care, 27, 1211-1212 (2004).
10. Cho et al., “Noninvasive Measurement of Glucose by Metabolic Heat Conformation Method”, Clinical Chemistry, 50, 1894-1898 (2004).
11. O. K. Cho and B. Holzgreve, “Process and device for non-invasive determination of glucose concentration in parts of the human body,” U.S. Pat. No. 5,795,305, Aug. 18, 1998.
12. O. K. Cho and B. Holzgreve, “Process and device for detecting the exchange of heat between the human body and the invented device and its correlation to the glucose concentration in human blood,” U.S. Pat. No. 5,924,996, Jul. 20, 1999.
13. O. K. Cho, Y. O. Kim, N. Sato, and H. Mitsumaki, “Optical measurement apparatus and blood sugar level measuring apparatus using the same,” United States Patent Application Publication 20060094941, May 4, 2006.

1-8. (canceled)

9. A method for estimating blood glucose variation in a patient based on metabolic parameters, comprising:
measuring raw metabolic parameters of the patient, wherein the raw metabolic parameters comprise conduction heat loss of an extremity of the patient, oxygen content of air expired by the patient, and a rate of expiration of air expired by the patient;
converting the raw metabolic parameters into metabolic features of the patient; and
calculating an estimated blood glucose variation for the patient based on the metabolic features.

10. The method of claim 9, wherein conduction heat loss is measured via a heat flow sensor.

11. The method of claim 10, wherein the heat flow sensor comprises a Data Harvest EasySense Advanced Datalogger™.

12. The method of claim 9, wherein oxygen content is measured via an oxygen analyzer.

13. The method of claim 12, wherein the oxygen analyzer comprises a Teledyne AX300 Oxygen Analyzer with R-17 MED Oxygen Sensor™.

14. The method of claim 9, wherein the rate of expiration of air is measured via a gas speed-measuring device.

15. The method of claim 14, wherein the gas speed-measuring device comprises a Vitalograph Micro Spirometer™.

16. The method of claim 9, wherein the calculating step comprises use of a classification model, wherein the classification model takes the metabolic features as inputs and provides the estimated blood glucose variation as an output.

17. The method of claim 16, wherein the classification model classifies the estimated blood glucose variation into one of up to five classes.

18. The method of claim 9, wherein the calculating step comprises use of a multiple regression analysis, wherein the multiple regression analysis is used to calculate the estimated blood glucose variation from the metabolic features.

19. An apparatus comprising one or more memory units having computer-useable instructions encoded thereon for performing a method of classifying blood glucose variation of a patient based on metabolic parameters, the method comprising:
receiving raw metabolic parameters of the patient;
converting, in a first computer process, the raw metabolic parameters into metabolic features of the patient;
computing, in a second computer process, variations between the metabolic features of the patient and stored metabolic features of the patient; and
classifying, via a third computer process, the blood glucose variation of the patient based on the computed variations.

20. The apparatus of claim 19, wherein the raw metabolic parameters comprise conduction heat loss of an extremity of the patient, oxygen content of air expired by the patient, and a rate of expiration of air expired by the patient.

21. The apparatus of claim 20, wherein the metabolic features comprise heat dissipation and a resting metabolic rate.

22. The apparatus of claim 19, wherein the third computer process comprises a regression analysis.

23. The apparatus of claim 22, wherein the third computer process comprises a multiple linear regression analysis.

24. The apparatus of claim 23, wherein the multiple linear regression analysis is performed using a least-squares method.

25. The apparatus of claim 19, wherein the third computer process comprises a linear discriminant analysis.

26. An apparatus comprising a heat flow sensor and a heat flow analyzer, which respectively are capable of measuring and computing the conduction heat loss at an extremity's tip.

27. The apparatus of claim 26, further comprising an oxygen analyzer, which is capable of measuring the percentage oxygen content of expired air from the mouth.

28. The apparatus of claim 26, further comprising a gas speed-measuring device, which is capable of measuring the expiratory flow rate at the mouth cavity.
